(19) United States
(12) Patent Application Publication
(10) Pub. No.: US 2022/0331003 A1
(43) Pub. Date: Oct. 20, 2022

(54) CABLE AND WIRE ROUTING IN A MECHANICAL ARM OF A SURGICAL APPARATUS

(71) Applicant: MEMIC INNOVATIVE SURGERY LTD, Or-Yehuda (IL)

(72) Inventors: Dvir COHEN, Ramot-Menashe (IL); Yaron LEVINSON, Tel-Aviv (IL); Yiftah NETA, Gilon (IL)

(21) Appl. No.: 17/640,358

(22) PCT Filed: Sep. 6, 2020
(86) PCT No.: PCT/IB2020/058292

§ 371 Date: Mar. 4, 2022

Related U.S. Application Data
(60) Provisional application No. 62/897,293, filed on Sep. 7, 2019.

(51) Int. Cl. A61B 18/14; A61B 34/35

(52) U.S. Cl. CPC A61B 18/1445; A61B 34/35

(57) ABSTRACT

An apparatus for performing electrosurgical operations using an electrosurgical power generator comprises an articulated mechanical arm, an electrosurgical grasper connected to the mechanical arm at a distal end thereof, a flexible sleeve at least partly disposed in a bendable portion of the arm, an outer surface of the sleeve comprising a plurality of surface features that define a helical wire-path around a central longitudinal axis of the sleeve, an actuation-cable passing through an inner conduit of the sleeve and mechanically coupled to the grasper to open and shut the grasper, and an electrically conductive wire for providing electrical connectivity from the power generator to the grasper, the wire being disposed on the outside of the sleeve and engaged with one or more of the surface features so as to follow therethrough the helical wire-path.

### CROSS-REFERENCE TO RELATED APPLICATIONS

This patent application claims the benefit of U.S. Provisional Patent Application No. 62/897,293 filed on Sep. 7, 2019, which is incorporated herein by reference in its entirety.

### FIELD OF THE INVENTION

The present invention relates to surgical and electrosurgical apparatuses used for gripping, coagulating The present invention relates to surgical and electrosurgical apparatuses used for gripping, coagulating, sealing, manipulating and/or cutting tissue. More particularly, it relates to electrosurgical apparatuses including mechanical arms, and devices for routing, in paths within and through such arms, mechanical cables and electrical wires needed for the operation of such apparatuses and arms. In particular, the present invention is suitable for separating and retaining mechanical cables and electrical wires in such paths within bendable sections of mechanical surgical arms.

### BACKGROUND

It is well established that there are benefits of minimally invasive surgery. Instruments for such surgery typically have a surgical end effector located at the distal end of an articulated surgical arm (preferably with minimum diameter) that is inserted through a small opening (e.g., body wall incision, natural orifice) to reach a surgical site. In some instances, surgical instruments can be passed through a cannula and an endoscope can be used to provide images of the surgical site.

Surgical instruments have been developed that utilize an end effector that integrates the use of tissue fusing and cutting for both convenience and cutting accuracy. In some cases, articulated surgical arms have one or more bending portions which are controlled with mechanical cables whose longitudinal movement affect bending and ultimately control the location of the end effector and change its orientation with reference to the surgical arm's longitudinal axis. In some case, the surgical arm is capable of retroflected bending relative to the surgical arm longitudinal axis.

The current state of the art is lacking devices and methods that can provide optimal routing of respective mechanical wires and electrical cables through lengths of surgical arms and especially bendable portions thereof, where the electrical wire is separated from the mechanical cable to avoid electrical and/or magnetic interference without stressing the electrical wire when portions of the arm are flexed, and where the electrical wire is neither under too much tension when a corresponding portion of the arm is bent to a small radius of curvature, nor given too much slack that can lead to fouling and misrouting. Such devices and methods would optimally retain the electric wires in surgical arms in safe and efficient routings therein.

### SUMMARY

According to embodiments disclosed herein, an apparatus for performing electrosurgical operations using an electrosurgical power generator comprises: (a) an articulated mechanical arm; (b) an electrosurgical grasper comprising a plurality of jaws and connected to the mechanical arm at a distal end thereof; (c) a flexible sleeve at least partly disposed in a bendable portion of the arm, an outer surface of the sleeve comprising a plurality of surface features that define a helical wire-path around a central longitudinal axis of the sleeve; (d) an actuation-cable passing through an inner conduit of the sleeve and mechanically coupled to the grasper to effect a movement of at least one jaw; and (e) an electrically conductive wire for providing electrical connectivity from the power generator to the grasper, the wire being disposed on the outside of the sleeve and engaged with one or more of the surface features so as to follow therethrough the helical wire-path.

In some embodiments, the plurality of surface features can comprise non-continuous protrusions, the helical wire-path passing therebetween.

In some embodiments, it can be that the plurality of surface features comprises alternating parallel longitudinal ribs and troughs, the ribs and troughs being helically aligned around the central longitudinal axis of the sleeve for at least a lengthwise portion of the sleeve, the helical wire-path passing within one of the troughs. In some such embodiments,

the plurality of surface features can comprise at least 3 ribs and at least 3 troughs. In some such embodiments, it can be that for a first lengthwise portion of the sleeve, the parallel ribs and troughs are parallel to the central longitudinal axis, and for a second lengthwise portion of the cable-sleeve the parallel ribs wind helically around the central axis.

In some embodiments, a helical pitch of the helical wire-path can have variability of less than ±50% along the length of the helical wire-path. In some embodiments, a helical pitch of the helical wire-path is either constant or has a variability of less than ±10% along the length of the helical wire-path.

In some embodiments, a helical pitch of the helical wire-path can be at least ⅓ of the length of a central-axis path of a corresponding portion of the sleeve. In some embodiments, a helical pitch of the helical wire-path is at least ½ of the length of a central-axis path of a corresponding portion of the sleeve. In some embodiments, a helical pitch of the helical wire-path is at most equal to 1.5 times the length of a central-axis path of a corresponding portion of the sleeve, or 1.25 times the length, or the length itself.

In some embodiments, a ratio between a helical pitch and a helical amplitude of the helical wire-path can be at least 10, or at least 20, or at least 50.

In some embodiments, the bendable portion of the arm can comprise a plurality of bendable segments. In some embodiments, the bendable portion of the arm can comprise non-contiguous segments.

In some embodiments, it can be that the sleeve is constrained to bend and/or straighten together with the bendable portion of the arm, and the path of the electric wire is maintained as helical relative to the bent and/or straightened path of the central longitudinal axis of the sleeve. In some such embodiments, the path of the electric wire can be maintained as helical relative to the bent and/or straightened path of the central axis of the sleeve, for any bending of the bendable portion of the arm to a radius of curvature greater than twice the diameter of the bendable portion. In some embodiments, the helical path of the electric wire can be maintained as helical relative to the bent and/or straightened path of the central axis of the sleeve, for any bending of the bendable portion of the arm to a radius of curvature greater than 1.5 times the diameter of the bendable portion.

In some embodiments, it can be that the bending and/or straightening of the sleeve assembly does not substantially increase tension in the wire.

In some embodiments, the inner conduit can have a circular cross-section. In some such embodiments, an inner diameter of the inner conduit can be at least 0.5 mm and at most 2.0 mm.

In some embodiments in which the surface features comprise helical ribs, a maximum helical amplitude of the ribs on the outer surface of the sleeve can be at least 1.0 mm and at most 2.0 mm. In some such embodiments, a maximum helical amplitude of the ribs on the outer surface of the sleeve can be at least 1.25 mm and at most 1.5 mm.

In some embodiments, the surface features can be formed integrally with the sleeve.

In some embodiments, the sleeve can comprise a thermoplastic elastomer that includes a rigid polyamide and a flexible polyether.

In some embodiments, the electrosurgical grasper can provide at least a bipolar electrosurgery mode when electrically connected to the electrosurgical power generator.

In some embodiments, the actuation-cable can be mechanically coupled to the grasper such that rotation of the actuation-cable about its central axis controls the movement of at the least one jaw. In some embodiments, the actuation-cable can be mechanically coupled to the grasper such that longitudinal motion of the actuation-cable within the arm controls the movement of at the least one jaw.

In some embodiments, the surgical apparatus can additionally comprise the electrosurgical power generator, and an electrically conductive wire can provide electrical connectivity from the power generator to the grasper.

According to embodiments disclosed herein, a surgical apparatus for use with a source of electrical power comprises: a. an articulated mechanical arm; b. a tool connected to the mechanical arm at a distal end thereof, the tool being electrically powered or having at least one electrically-powered auxiliary device mounted thereto; c. a flexible sleeve at least partly disposed in a bendable portion of the arm, an outer surface of the sleeve comprising a plurality of surface features that define a helical wire-path around a central longitudinal axis of the sleeve; and d. an electrically conductive wire for providing electrical connectivity from the electrical power source to the tool or to the mounted auxiliary device, the wire being disposed on the outside of the sleeve and engaged with one or more of the surface features so as to follow therethrough the helical wire-path.

In some embodiments, the tool is a surgical tool.

In some embodiments, the surgical tool is selected from the group consisting of a grasper, forceps, scissors, a clamp, a hook and a laser.

In some embodiments, is the tool and/or the auxiliary device is a camera—i.e. the electrically conductive wire being for providing electrical connectivity from the electrical power source to the camera.

In some embodiments, the tool and/or the auxiliary device is a data-acquisition tool—the electrically conductive wire for providing electrical connectivity from the electrical power source to the data-acquisition tool.

In some embodiments, the data-acquisition tool is or comprises at least one of an electrically-powered thermometer, a camera, and an electrically-powered microphone.

In some embodiments, the tool and/or the auxiliary device comprises an electrically-powered illumination source (for example, a light-emitting-diode (LED))—i.e. the electrically conductive wire for providing electrical connectivity from the electrical power source to the illumination source.

In some embodiments, the tool has at least one internal degree of freedom.

In some embodiments, additionally comprising an actuation-cable passing through an inner conduit of the sleeve and mechanically coupled to the tool so as to: (i) modify an internal configuration of the tool with respect to one or more of the degrees of freedom; and/or (ii) mechanically operate or actuate the tool.

In some embodiments, the plurality of surface features comprises non-continuous protrusions, the helical wire-path passing therebetween.

In some embodiments, the plurality of surface features comprises alternating parallel longitudinal ribs and troughs, the ribs and troughs being helically aligned around the central longitudinal axis of the sleeve for at least a lengthwise portion of the sleeve, the helical wire-path passing within one of the troughs.

In some embodiments, the plurality of surface features comprises at least 3 ribs and at least 3 troughs.

In some embodiments, for a first lengthwise portion of the sleeve, the parallel ribs and troughs are parallel to the central longitudinal axis, and for a second lengthwise portion of the cable-sleeve the parallel ribs wind helically around the central axis.

In some embodiments, a helical pitch of the helical wire-path has a variability of less than ±50% along the length of the helical wire-path.

In some embodiments, a helical pitch of the helical wire-path is either constant or has a variability of less than ±10% along the length of the helical wire-path.

In some embodiments, a helical pitch of the helical wire-path is at least ⅓ of the length of a central-axis path of a corresponding portion of the sleeve.

In some embodiments, a helical pitch of the helical wire-path is at least ½ of the length of a central-axis path of a corresponding portion of the sleeve.

In some embodiments, a helical pitch of the helical wire-path is at most equal to 1.5 times the length of a central-axis path of a corresponding portion of the sleeve, or 1.25 times the length, or the length itself.

In some embodiments, a ratio between a helical pitch and a helical amplitude of the helical wire-path is at least 10, or at least 20, or at least 50.

In some embodiments, the bendable portion of the arm comprises a plurality of bendable segments.

In some embodiments, the bendable portion of the arm comprises non-contiguous segments.

In some embodiments, the sleeve is constrained to bend and/or straighten together with the bendable portion of the arm, and the path of the electric wire is maintained as helical relative to the bent and/or straightened path of the central longitudinal axis of the sleeve.

In some embodiments, the path of the electric wire is maintained as helical relative to the bent and/or straightened path of the central axis of the sleeve, for any bending of the bendable portion of the arm to a radius of curvature greater than twice the diameter of the bendable portion.

In some embodiments, the helical path of the electric wire is maintained as helical relative to the bent and/or straightened path of the central axis of the sleeve, for any bending of the bendable portion of the arm to a radius of curvature greater than 1.5 times the diameter of the bendable portion.

In some embodiments, the bending and/or straightening of the sleeve assembly does not substantially increase tension in the wire.

In some embodiments, the inner conduit has a circular cross-section.

In some embodiments, an inner diameter of the inner conduit is at least 0.5 mm and at most 2.0 mm.

In some embodiments, a maximum helical amplitude of the ribs on the outer surface of the sleeve is at least 1.0 mm and at most 2.0 mm.

In some embodiments, a maximum helical amplitude of the ribs on the outer surface of the sleeve is at least 1.25 mm and at most 1.5 mm.

In some embodiments, the surface features are formed integrally with the sleeve.

In some embodiments, the sleeve comprises a thermoplastic elastomer that includes a rigid polyamide and a flexible polyether.

A teleoperated robotic surgical system comprising: any apparatus disclosed herein; a patient side console configured to interface with the surgical tool to actuate the surgical tool to perform one or more surgical procedures; and a surgeon side console comprising one or more input devices configured to be manipulated by a surgeon and to transmit signals to control the surgical tool at the patient side console.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. Also, in some drawings the relative sizes of objects, and the relative distances between objects, may be exaggeratedly large or small for the sake of convenience and clarity of presentation. In the drawings:

FIG. 1 shows a simplified schematic of a surgical system according to embodiments of the present invention.

FIG. 2A shows a surgical arm unit according to embodiments of the present invention.

FIG. 2B shows a mechanical arm having a bendable portion comprising stacked links and end effector tooling, according to embodiments of the present invention.

FIG. 2C shows a result of bending the bendable portion of a mechanical arm, according to embodiments of the present invention.

FIG. 2D shows a schematic illustrating a central axis of a mechanical arm, a diameter of a section of mechanical arm, and a radius of curvature for a bendable portion of a mechanical arm as used in this disclosure.

FIG. 3 is a longitudinal cross-sectional projection of part of a mechanical arm, showing a cable- and wire-routing sleeve according to embodiments of the present invention.

FIG. 4 schematically shows the bent mechanical arm of FIG. 2C with an overlay of a similarly bent cable sleeve according to embodiments of the present invention.

FIGS. 5A and 5B schematically show a sleeve and its partial magnification having helical ribs and troughs as surface features, according to embodiments of the present invention,

FIG. 6 shows a sleeve having protrusions as surface features, according to embodiments of the present invention.

FIG. 7 shows the sleeve of FIG. 5A with a wire in a helical wire-path, according to embodiments of the present invention.

FIG. 8A is a two-dimensional projection of the wire of FIG. 7.

FIGS. 8B and 8C are respective two-dimensional projections of wires having different helical pitches.

FIG. 9 shows a schematic illustrating helical pitch and helical amplitude as used in this disclosure.

FIG. 10 shows an embodiment of a sleeve having straight ribs and troughs on the outer surface of a first portion of the length of the sleeve, and helical ribs and troughs on the outer surface of a second portion thereof, according to embodiments of the present invention.

### DETAILED DESCRIPTION OF THE EMBODIMENTS

According to embodiments, a mechanical surgical arm can house a ‘mechanical’ or ‘actuation’ cable for actuating and controlling the movements of a surgical tool located at the distal end of the arm. An example of a surgical tool is a multi-jaw grasper used in electrosurgery. The grasper preferably provides at least a bipolar electrosurgery mode typically used to fuse tissue (e.g. blood vessels) or for general coagulation of tissue bundles, cold cutting, tissue dissection, and tissue manipulation/retraction. A grasper can also provide a monopolar electrosurgery mode.

The ‘distal’ end of the arm is defined herein as the end to which the surgical tool is connected, i.e., the end which in normal surgical operation is furthest from an operator or user of a surgical apparatus comprising the arm. The term ‘mechanical’ is used herein to denote that the cable is generally not used for conducting electricity, and generally does not carry data, although in some embodiments the ‘mechanical’ cable can be used for carrying data and/or as a ground return to complete an electric circuit.

A mechanical or actuation wire can run the entire length of the arm or a portion thereof, and can be connected, directly or indirectly, at or near a proximal end of the arm to one or more mechanical and/or electronic control elements, such as a gearing or an actuator. The surgical tool may be controlled, directly or indirectly, by longitudinal force applied by the actuation cable (i.e., producing back-and-forth movements), or by rotation torque applied via the cable, depending upon the mechanical arrangements of the arm and/or the tool. It can be desirable to route the actuation cable through a longitudinal conduit, such as a tube or sleeve, deployed around a central axis of the arm so as to maintain a straightforward routing path thereby allowing maximum control of movement.

A mechanical surgical arm can additionally house an electric wire that—in an operating state of the surgical apparatus—conducts electricity from a power source such as (but not limited to) an electrosurgical generator to the same surgical tool controlled by the actuation cable. The wire can be single or double wire (or even triplex wire). If a single wire is used then an electric circuit can be completed by using another return path, e.g., a metallic arm housing or the actuation cable. It can be desirable to physically separate the path of the electric wire from that of the actuation cable to avoid, inter alia, potential tangling that could interfere with the functionality of the actuation cable and/or endanger the physical integrity of the less-robust electric wire. The electric wire is preferably both physically separated and electrically insulated from the actuation cable, which is preferably coaxial with the central axis of the arm for maximum function. Thus, the electric wire, if it were to follow a straight path through the arm, would necessarily be off-center. Retaining the wire in an off-center routing path can require a complicated mechanical scheme and, in some cases, may put excess strains, stresses and/or shear forces, even resulting in tearing, on the portion of the wire that passes through a bendable portion of the arm especially when the arm is placed in repeated retroflected position.

Therefore, it can be desirable to wrap the electric wire around the sleeve or tube through which the actuation cable passes, and, according to embodiments disclosed herein, use surface features provided of the outside surface of the sleeve to create a stable wire-path around the outside surface. A helical wire-path around the outside surface of the sleeve can be desirable since a central axis of the helix can be made to be coaxial with the central axis of the sleeve through which the actuation cable passes in the center of the arm. As will be discussed hereinbelow, the central axes of the sleeve and of the arm remain coaxial with each other, or at least parallel to each other if not coaxial, with the bending and straightening of the bendable portion of the arm; although seemingly distorted by the bending, the helical wire-path remains helical with respect to the path of the central axis of the sleeve. The bending of the bendable portion of the arm can include ‘extreme’ bending such as retroflex configurations and S-shaped curves, and the sleeve remains coaxial with (or parallel to) the arm in any such usage. Creating a stable helical wire-path centered around the central axis of the arm can have the advantage of reducing tension in the wire when the wire-path is bent along with the bendable portion of the arm, and/or reducing the amount of slack provided in the wire when assembled. A wire can slide longitudinally within its helical wire-path so that ‘excess’ wire in the ‘inside’ of a curve or bend can slide towards the ‘outside’ of the curve or bend where the wire would otherwise be under greater tension, or even become stretched or broken. This sliding helical movement of the electric wire within its helical wire-path is preferably unimpeded, or at least sufficiently unimpeded so that the bending and/or straightening of the sleeve assembly does not substantially increase tension in the wire. As a counter-example, if the wire were to be wrapped or tied around a surface feature of the outside surface of the sleeve, this sliding would most likely be overly impeded and tension could substantially increase in a portion of the wire, such as a portion that in an operating state is located on the ‘outside’ of a curve or bend.

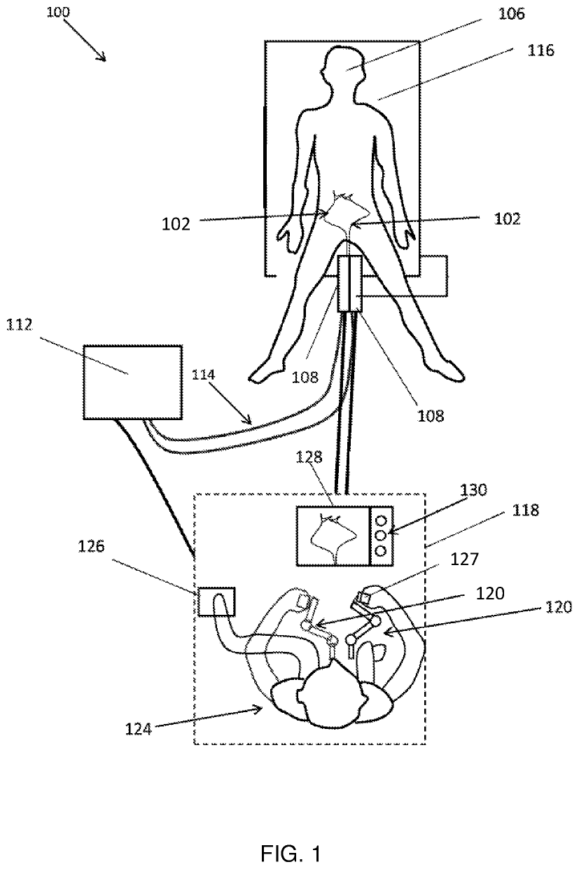

FIG. 1 shows a schematic illustration of a typical prior-art surgical system 100 according to embodiments. The system 100 includes two surgical mechanical arms 102. In other embodiments, a single surgical arm is provided. Surgical mechanical arms 102 are preferably sized and/or shaped for insertion into a human body or patient 106. Each of the surgical mechanical arms 102 is actuated by a respective motor unit 108. The surgical arms 102 and/or motor units 108 are supported in this example by attachment to a patient support 116, e.g., a bed but may also be supported by a patient side cart.

Power to the arms 102 and the motor units 108 is supplied by an electrosurgical generator 112. As known in the art of electrosurgery, an electrosurgical generator supplies high-frequency (e.g. radio frequency) alternating polarity, electric current. An electrosurgical generator 112 can be configured to supply different frequencies and/or power levels, for example, suitable for cutting and/or coagulating and/or sealing and/or desiccating and/or fulgurating tissue. An example of a suitable, commercially available electrosurgical generator 112 is a Covidien Force FX ESU Electrosurgical Generator. Power is supplied to the motor units 108 via cable/s 114 which are configured to transfer radio frequency electrosurgical power.

Movement and/or electrosurgical charging of the surgical arms 102 is/are controlled at a control console 118. Control console 118 includes a plurality of user interfaces including one or more of: input devices, e.g., input device arms 120 where the control console is configured to generate control signals based upon movement of the input device arms 120; touch-screen display 128 configured to receive user input and/or display imaging of a surgical zone, for example, to display images collected by a camera inserted into patient 106 using one of surgical arms 102; and one or more additional user interfaces 130 (e.g. button, switch, etc.).

Control console 118 includes a processor (not shown) configured to receive signals from a user input's and to send control signals to motor units 108 and/or electrosurgical generator 112. Foot pedal 126 and/or electrosurgical generator 112 include/s a processor (not shown) configured to receive control signals (e.g. generated by a user pressing on a portion/s of the foot pedal 126) to vary electrical power supplied to motor units 108 based on the control signals. Foot pedal control signals do not necessarily pass through a control unit processor.

The movement of input device arms 120 controls movement of a respective surgical device arm 102. A user 124 can position and/or move an input arm 120 by grasping an input device arm handle 127.

It can be desirable that a surgical arm be sized and/or shaped for insertion into a human body. For example, an arm can be sized and/or shaped for insertion through a laparoscopic port and/or for performing laparoscopic surgery. For example, an arm can be sized and/or shaped for insertion through a natural body orifice, e.g. vagina, anus, trachea, esophagus, ear canal.

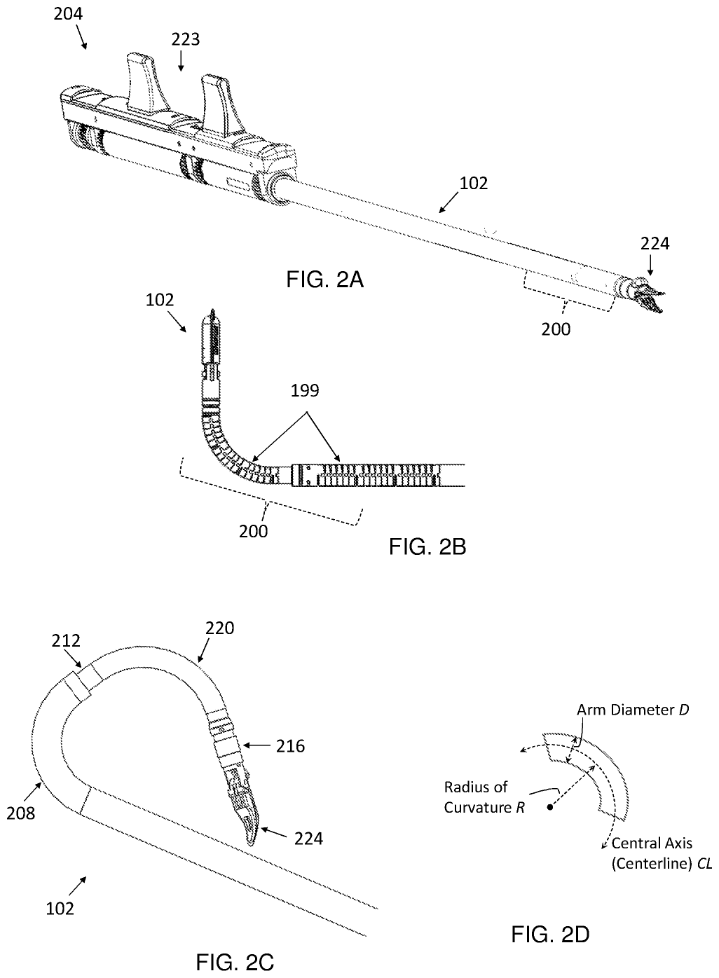

Referring now to FIG. 2A, arm unit 204 includes a proximal end where a support unit 223 is attached to an arm 102, and a distal end where an electrosurgical tool 224 such as the illustrated multi-jaw grasper is attached to the arm 102. The example of a multi-jaw grasper is intended to be non-limiting, and any appropriate surgical tool may be used. Various non-limiting examples of grasper tool designs are illustrated in the attached figures; any electrically powered surgical tool actuated by an actuation cable can be suitable for use as electrosurgical tools in the disclosed embodiments. A bendable portion 200 of the arm 102 is located along the length of the arm, closer to the distal end. The bendable portion 200 can comprise a series of stacked links 199 that provide external flexibility for the arm 102; an example of a plurality of stacked links 199 in a bendable portion 200 of an arm 102 is illustrated in FIG. 2B. As shown in FIG. 2B, an arm 102 or one or more segments of a bendable portion 200 can have different diameters while sharing the same central longitudinal axis.

The bendable portion 200 of an arm 102 can comprise non-contiguous segments. In other words, the bendable portion 200 can actually comprise multiple bendable portions with or without non bendable segments interposed therebetween. FIG. 2C illustrates a typical arm 102 bent in multiple locations along the bendable portion 200. A clockwise ‘tour’ of FIG. 2C reveals: rigid portion 202 (the most-proximal part of the arm 102 illustrated in FIG. 2C); a bendable segment 208 comprising a first bendable segment within the length of bendable portion 200; a rigid connecting segment 212; a bendable segment 220 that comprises a second bendable segment within the length of bendable portion 200; and a rigid segment 216 to which the surgical tool 224 is connected. Rigid segment 216 may house mechanical arrangements for translating control movements of an actuation cable to the surgical tool 224.

Any portion or segment in the bendable portion 200 of the arm 102 can be bent to a radius of curvature R, which for the purposes of this disclosure is calculated as the radius of curvature of the central axis, or centerline CL, as illustrated for the sake of clarity in FIG. 2D. Lower limits on the radius of curvature R, in embodiments, can be defined by the size and specific design of the bendable portion and its component links, as well as by the diameter D of the arm 102. For example, the radius of curvature R can be limited to being at least 3 times the diameter of the arm 102, or at least twice the diameter of the arm 102, or at least 1.5 times the diameter of the arm, or at least 1.25 times the diameter of the arm 102.

The diameter D of a bendable portion 200 (or any segment thereof) of a mechanical surgical arm 102 suitable for electrosurgery . . . and especially suitable for minimally invasive surgery can be in the range of 6 to 12 mm, or 7 to 11 mm, or 8 to 10 mm, or 8 to 9 mm. Different segments can be designed to have different D values. The ‘length’ of a link 199, i.e., when incorporated in a bendable portion 200 that is not bent, can be in the range of 1.5 to 4 mm, or 2.0 to 3.25 mm, or 2.25 to 2.75 mm. Each link 199, when the corresponding bendable portion 200 (or segment thereof) is maximally flexed or bent, can correspond to an arc of 5° to 15°, or 6° to 13°, or 7° to 11°, or 8° to 10°. The resulting radius of curvature, R, can be in the range of 10 to 20 mm, or 11 to 16 mm, or 12 to 15 mm, or 13 to 14 mm.

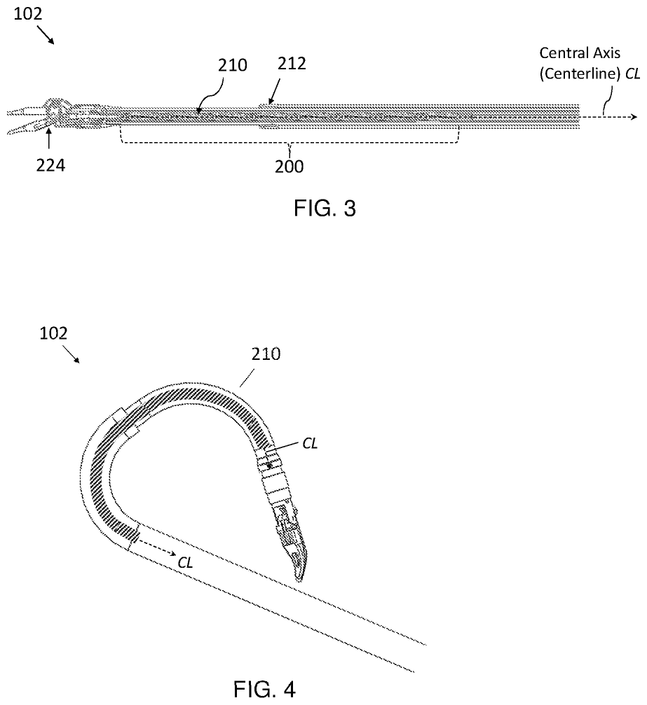

Referring now to FIG. 3, which shows a cross-sectional view of the distal portion of an arm 102. A cable- and wire-routing sleeve 210, not in cross-section, is shown as being disposed within the bendable portion 200 of an arm 102.

Use of the term “disposed within” throughout this disclosure and the appended claims should be understood to include, interchangeable, either one of “entirely disposed within” or “partially disposed within”. For example, whilst FIG. 3 shows the entire length of sleeve 210 as being subsumed in the marked length of bendable portion 200, the sleeve 210 of FIG. 3 would still be “disposed within” the bendable portion 200 even if there were an overlap between the length of the sleeve 210 and the length of the bendable portion 200 such that either one or both ends of the sleeve were to extend from the marked length of the bendable portion 200. Moreover, the length marked as the bendable portion 200 can include, as discussed in connection with FIG. 2C, one or more rigid sections, e.g., rigid section 212. The purpose, function, shape and manufacture of sleeve 210 will be further discussed below. It can be desirable for the sleeve to have a central longitudinal axis that is coaxial with the central longitudinal axis CL of the arm 102, as is shown in FIG. 3.

FIG. 4 illustrates schematically the effect of the bending of the bendable portion 200 of the arm 102 on the sleeve 210. The sleeve 210 will obviously bend within the confines of the bent bendable portion 200. It is desirable, however, that the sleeve 210 be suitably constrained within the arm, and be sufficiently flexible, so that its central axis and the central axis CL of the arm remain coaxial (i.e., parallel) throughout the range of bending associated with the ranges of radius of curvature R of the bendable portion 200 as described hereinabove. While FIG. 4 appears to show a single sleeve 210 extending the length of the bendable portion 200 of the arm 102, in some embodiments there can more than one sleeve 210; for example, there can be one sleeve for each bendable segment (e.g., segments 208 and 220) of the bendable portion 200. A thermoplastic elastomer can be chosen with physical properties that enable repeated bending and straightening as described here. The inventors have found that suitable material for fabricating a sleeve 210 is a thermoplastic elastomer that includes a rigid polyamide and a flexible polyether, an example of which is a PEBAX® formulation available commercially from Arkema of Colombes Cedex, France. The sleeve can be manufactured using any of the methods known in the art, including, without limitation, molding, extrusion and 3D printing. Additionally or alternatively, a sleeve having helically disposed surface features can be fabricated from an ‘untwisted’ sleeve having surface features that are not already helically disposed.

A sleeve can be deployed within an arm to route a mechanical cable, such as an actuation cable used to transfer or produce movements in a surgical tool connected to the distal end of the arm, and at the same time to route an electrical wire, such as a wire that provides electrical connectivity between a power source and the surgical tool. According to embodiments, a sleeve includes an inner, or central longitudinal conduit through which the mechanical cable passes, and a plurality of surface features on an outer surface of the sleeve through which the electric wire can be routed, where the surface features can be useful for retaining the wire within a path, such as a helical path.

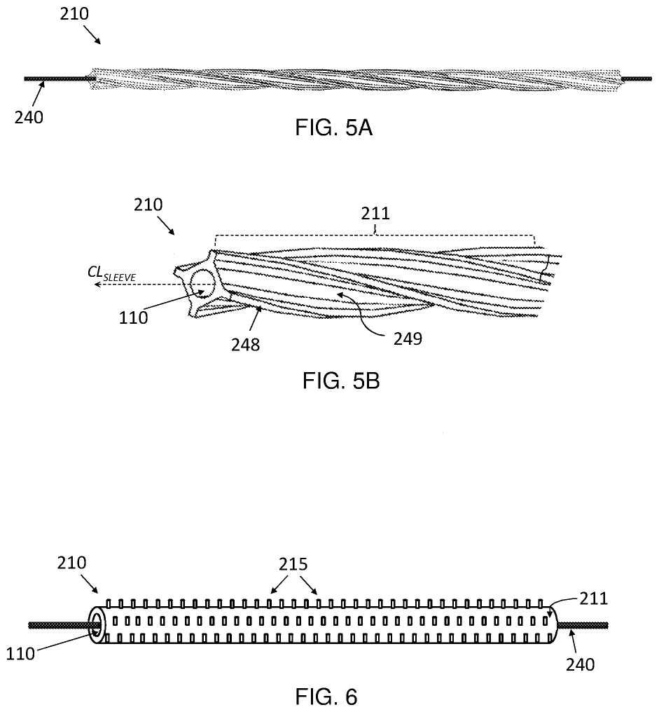

A non-limiting example of a sleeve 210 having an inner conduit 110 and surface features on its outer surface 211 is shown in FIGS. 5A and 5B. The inner conduit 110 can have a circular cross-section; this not only facilitates having a regularly helical wire-path on the outer surface of the sleeve 210, the circular-cross-section is also beneficial in assembly and operation of the mechanical arm 102. For example, a sleeve 210 comprising a circular-cross-section inner conduit 110 need not be oriented in a specific way during assembly, and the sleeve 210 can be bent, etc. with equal ease (force) in every direction. A different shape could complicate assembly, and/or affect the bending behavior of the sleeve 210. As shown, in FIG. 5A, actuation cable 240 passes through the inner conduit 110. The exemplary surface features illustrated in FIGS. 5A and 5B include parallel helical ribs 248 alternating with helical troughs 249. The ribs 248 and troughs 249 are formed so as to wrap helically around a central longitudinal axis CLSLEEVE of the sleeve 210 and run the entire length of the outer surface 211. In some embodiments, the ribs 248 and troughs 249 run at least 70% or at least 80% or at least 90% of the length of the outer surface 211. As will be described below, a wire (e.g., a segment of a wire) can be retained in a helical wire-path defined by one of the troughs with the wire being subjected to, at most, a moderate amount of tension. The illustrated examples accompanying this disclosure uniformly show 4 ribs and 4 troughs. However, any reasonable number of ribs and troughs can be implemented as surface features, from a minimum of 3 to as many as 6 or 8 (or even 10) ribs and troughs. If there are too few ribs, i.e., fewer than 3, then the wire may not be retained in a helical path; if there are too many ribs, then there might not be enough room in a trough for the wire, depending on the physical dimensions of the wire. Although not shown in the figures, one or more troughs 249 not occupied by the electric wire can be used as paths for additional cables—mechanical, data, and/or electrical.

Another non-limiting example of a sleeve having an inner conduit 110 and surface features on its outer surface 211 is shown in FIG. 6. In this example, the surface features include a plurality of protrusions 215. A wire can be retained in a helical wire-path by threading the wire between the appropriate protrusions 215. In another example (not shown) the protrusions 215 can have mushroom-like ‘caps’ to better retain the wire in a helical wire-path. In yet another example (also not shown), it can be that only those protrusions 215 needed to define a helical wire-path are provided.

The number of wire wrappings, or wrappings per unit of length (i.e., the length of either the wire or the central axis), needs to be limited so as to avoid producing a magnetic field that could interfere with proper operation of the arm and/or of the surgical tool, or even magnetize the actuation cable. The advantages, as described above, of the helical wrapping of the wire within a stable helical wire-path, can be realized with a small number of wrappings. For example, a sleeve with a length of 100 mm may be wrapped with no more than 3 helical revolutions, or no more than 2, or even fewer. Thus, the helical pitch—whether for a single sleeve passing the entire bendable portion of the arm, or for any one sleeve or portion thereof, of multiple sleeves in multiple segments of the bendable portion of the arm—can be at least ⅓ of the length of a central-axis path of a corresponding portion of the sleeve, or at least ½ of the length. In some embodiments, at least one full helical revolution of the wire is necessary to allow in-wire-path helical sliding and other advantages including avoiding electrical resonance of employing a helical wire-path, and in other embodiments four-fifths, three-quarters, or even two-thirds of a revolution can be sufficient. In some embodiments, the helical pitch is at most equal to 1.5 times the length of a central-axis path of a corresponding portion of the sleeve, or at most 1.25 times the length, or at most the length itself.

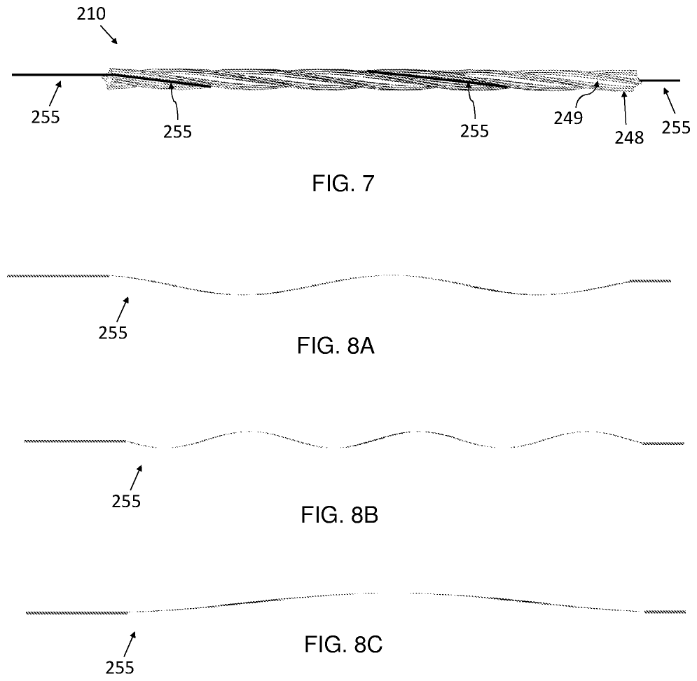

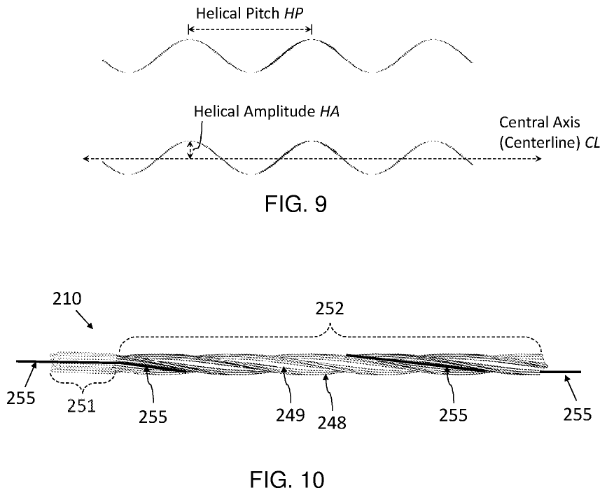

Referring now to FIG. 7, the sleeve 210 of FIGS. 5A and 5B is shown with a wire 255 disposed (in corresponding part) in a helical wire-path in one of the troughs 249. As discussed above, a moderate number of wrappings of a wire is preferable to avoid producing a potentially problematic magnetic field around the actuation cable 240. In the particular example of FIG. 7, the helical pitch is such that there are fewer than two wrappings of the wire around this particular sleeve. (The usage of the terms helical pitch and helical amplitude as used in this disclosure, and in the appended claims, are illustrated for added clarity in FIG. 9.) The helical pitch employed preferably has at most a moderate amount of variability along the length of the sleeve 210, for example variability of less than ±50%, or less than ±10% or 0%, i.e., remains constant (where ‘constant’ means within a tolerance of ±2%).

The actual shape of the wire 255 of FIG. 7 is shown in two-dimensional projection in FIG. 8A. Here it is easier to see that there are approximately (but not quite) two wrappings of the wire 255 around the sleeve 210. Since, as shown in FIG. 9, helical pitch HP is defined (in this disclosure, as well as in normal scientific usage) as the linear distance between adjacent ‘peaks’ of a helix, it can be said that the helical pitch HP of wire 255 in FIG. 8A is slightly more than half the central-axis (CL) length of the corresponding sleeve 210. In other words, if the sleeve 210 of FIG. 7 were 100 mm in length, the corresponding value of HP would be more than 50 mm Examples of wires 255 with alternative helical pitches HP are shown (in two-dimensional projection) in FIGS. 8B and 8C. The wire 255 in FIG. 8B has a helical pitch equal to about one-third of the length of the sleeve 210, while the wire 255 in FIG. 8C has a helical pitch roughly equal to the length of the sleeve 210. It will be obvious to the skilled artisan that the wires 255 of FIGS. 8B and 8C would require a sleeve 210 with differently formed ribs 248 and troughs 249 than the sleeve 210 of FIGS. 5A, 5B and 7—or, alternatively, a sleeve 210 with alternative surface features such as, for example, the protrusions 215 of FIG. 6.

FIG. 9 illustrates the terms helical pitch HP and helical amplitude HA as used herein. HP, as mentioned earlier, is the linear distance between successive helical peaks. HA is the distance (height) of a helical loop above the central axis CL of the helix. A sleeve 210 having ribs 248 and troughs 249 as illustrated, inter alia, in FIG. 7, can have an HA value between 1.0 and 2.0 mm, between 1.1 and 1.7 mm, or between 1.25 and 1.5 mm. A ratio between a helical pitch HP and helical amplitude HA for a helically-ribbed sleeve can be at least 10, at least 20, or at least 50.

FIG. 10 illustrates an embodiment of a sleeve 210 in which the parallel alternating ribs 248 and troughs 249 are ‘straight’, i.e., parallel to a central axis of the sleeve, for a first portion of the sleeve length, and ‘helical’ for a second portion of the sleeve length. Such an embodiment may be particularly suitable for use in mechanical arms when one or both of the ends of the sleeve are not housed in a bendable portion of the arm but, instead, in a rigid section adjacent to the bendable portion.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

Any feature or combination of features described in the present document may be combined with any feature or combination of features described in U.S. patent application Ser. No. 15/915,237 filed on Mar. 8, 2018 and published as US Patent Publication US20180256246A1; and U.S. patent application Ser. No. 15/454,123 filed on Mar. 9, 2017 and published as US Patent Publication US20170258539A1; U.S. patent application Ser. No. 15/501,862 filed on Feb. 6, 2017 and published as US Patent Publication US20170239005A1; all of which are hereby incorporated by reference herein as if fully set forth in their entirety.

In the description and claims of the present disclosure, each of the verbs, “comprise”, “include” and “have”, and conjugates thereof, are used to indicate that the object or objects of the verb are not necessarily a complete listing of members, components, elements or parts of the subject or subjects of the verb. As used herein, the singular form “a”, “an” and “the” include plural references unless the context clearly dictates otherwise. For example, the term “a marking” or “at least one marking” may include a plurality of markings.

1. A surgical apparatus for use with a source of electrical power, the surgical apparatus comprising:
a. an articulated mechanical arm;
b. a tool connected to the mechanical arm at a distal end thereof, the tool being electrically powered or having at least one electrically-powered auxiliary device mounted thereto;
c. a flexible sleeve at least partly disposed in a bendable portion of the arm, an outer surface of the sleeve comprising a plurality of surface features that define a helical wire-path around a central longitudinal axis of the sleeve; and
d. an electrically conductive wire for providing electrical connectivity from the electrical power source to the tool or to the mounted auxiliary device, the wire being disposed on the outside of the sleeve and engaged with one or more of the surface features so as to follow therethrough the helical wire-path.

2. The surgical apparatus of claim 1 wherein the tool is a surgical tool.

3. The surgical apparatus of claim 2, wherein the surgical tool is selected from the group consisting of a grasper, forceps, scissors, a clamp, a hook and a laser.

4. The surgical apparatus of any one of claims 1 to 3, wherein is the tool and/or the auxiliary device is a camera, the electrically conductive wire being for providing electrical connectivity from the electrical power source to the camera.

5. The surgical apparatus of any one of claims 1 to 4, wherein the tool and/or the auxiliary device is a data-acquisition tool.

6. The surgical apparatus of claim 5, wherein the data-acquisition tool is or comprises at least one of an electrically-powered thermometer, a camera, and an electrically-powered microphone.

7. The surgical apparatus of any one of claims 1 to 6, wherein is the tool and/or the auxiliary device comprises an electrically-powered illumination source.

8. The surgical apparatus of any one of claims 1 to 7, wherein the tool has at least one internal degree of freedom.

9. The surgical apparatus of any one of claims 1 to 8, additionally comprising an actuation-cable passing through an inner conduit of the sleeve and mechanically coupled to the tool so as to: (i) modify an internal configuration of the tool with respect to one or more of the degrees of freedom; and/or (ii) mechanically operate or actuate the tool.

10. The surgical apparatus of any one of claims 1 to 9, wherein the plurality of surface features comprises non-continuous protrusions, the helical wire-path passing therebetween.

11. The surgical apparatus of any one of claims 1 to 10, wherein the plurality of surface features comprises alternating parallel longitudinal ribs and troughs, the ribs and troughs being helically aligned around the central longitudinal axis of the sleeve for at least a lengthwise portion of the sleeve, the helical wire-path passing within one of the troughs.

12. The surgical apparatus of claim 11, wherein the plurality of surface features comprises at least 3 ribs and at least 3 troughs.

13. The surgical apparatus of either one of claim 11 or 12, wherein for a first lengthwise portion of the sleeve, the parallel ribs and troughs are parallel to the central longitudinal axis, and for a second lengthwise portion of the cable-sleeve the parallel ribs wind helically around the central axis.

14. The surgical apparatus of any one of claims 1 to 13, wherein a helical pitch of the helical wire-path has a variability of less than ±50% along the length of the helical wire-path.

15. The surgical apparatus of any one of claims 1 to 13, wherein a helical pitch of the helical wire-path is either constant or has a variability of less than ±10% along the length of the helical wire-path.

16. The surgical apparatus of any one of claims 1 to 15, wherein a helical pitch of the helical wire-path is at least ⅓ of the length of a central-axis path of a corresponding portion of the sleeve.

17. The surgical apparatus of any one of claims 1 to 15, wherein a helical pitch of the helical wire-path is at least ½ of the length of a central-axis path of a corresponding portion of the sleeve.

18. The surgical apparatus of any one of claims 1 to 15, wherein a helical pitch of the helical wire-path is at most equal to 1.5 times the length of a central-axis path of a corresponding portion of the sleeve, or 1.25 times the length, or the length itself.

19. The surgical apparatus of any one of claims 1 to 18, wherein a ratio between a helical pitch and a helical amplitude of the helical wire-path is at least 10, or at least 20, or at least 50.

20. The surgical apparatus of any one of claims 1 to 19, wherein the bendable portion of the arm comprises a plurality of bendable segments.

21. The surgical apparatus of any one of claims 1 to 20, wherein the bendable portion of the arm comprises non-contiguous segments.

22. The surgical apparatus of any one of claims 1 to 21, wherein the sleeve is constrained to bend and/or straighten together with the bendable portion of the arm, and the path of the electric wire is maintained as helical relative to the bent and/or straightened path of the central longitudinal axis of the sleeve.

23. The surgical apparatus of claim 22, wherein the path of the electric wire is maintained as helical relative to the bent and/or straightened path of the central axis of the sleeve, for any bending of the bendable portion of the arm to a radius of curvature greater than twice the diameter of the bendable portion.

24. The surgical apparatus of claim 23, wherein the helical path of the electric wire is maintained as helical relative to the bent and/or straightened path of the central axis of the sleeve, for any bending of the bendable portion of the arm to a radius of curvature greater than 1.5 times the diameter of the bendable portion.

25. The surgical apparatus of any one of claims 1 to 24, wherein the bending and/or straightening of the sleeve assembly does not substantially increase tension in the wire.

26. The surgical apparatus of any one of claims 1 to 25, wherein the inner conduit has a circular cross-section.

27. The surgical apparatus of any one of claims 1 to 26, wherein an inner diameter of the inner conduit is at least 0.5 mm and at most 2.0 mm.

28. The surgical apparatus of any one of claims 11 to 27, wherein a maximum helical amplitude of the ribs on the outer surface of the sleeve is at least 1.0 mm and at most 2.0 mm.

29. The surgical apparatus of any one of claims 11 to 27, wherein a maximum helical amplitude of the ribs on the outer surface of the sleeve is at least 1.25 mm and at most 1.5 mm.

30. The surgical apparatus of any one of claims 1 to 29, wherein the surface features are formed integrally with the sleeve.

31. The surgical apparatus of any one of claims 1 to 30, wherein the sleeve comprises a thermoplastic elastomer that includes a rigid polyamide and a flexible polyether.

32. A teleoperated robotic surgical system comprising: the apparatus of any one of claims 1 to 31; a patient side console configured to interface with the surgical tool to actuate the surgical tool to perform one or more surgical procedures; and a surgeon side console comprising one or more input devices configured to be manipulated by a surgeon and to transmit signals to control the surgical tool at the patient side console.

33. An apparatus for performing electrosurgical operations using an electrosurgical power generator, the apparatus comprising:
a. an articulated mechanical arm;
b. an electrosurgical grasper comprising a plurality of jaws and connected to the mechanical arm at a distal end thereof;
c. a flexible sleeve at least partly disposed in a bendable portion of the arm, an outer surface of the sleeve comprising a plurality of surface features that define a helical wire-path around a central longitudinal axis of the sleeve;
d. an actuation-cable passing through an inner conduit of the sleeve and mechanically coupled to the grasper to effect the movement of at least one jaw; and
e. an electrically conductive wire for providing electrical connectivity from the power generator to the grasper, the wire being disposed on the outside of the sleeve and engaged with one or more of the surface features so as to follow therethrough the helical wire-path.

34. The apparatus of claim 33, wherein the plurality of surface features comprises non-continuous protrusions, the helical wire-path passing therebetween.

35. The apparatus of claim 33, wherein the plurality of surface features comprises alternating parallel longitudinal ribs and troughs, the ribs and troughs being helically aligned around the central longitudinal axis of the sleeve for at least a lengthwise portion of the sleeve, the helical wire-path passing within one of the troughs.

36. The apparatus of claim 35, wherein the plurality of surface features comprises at least 3 ribs and at least 3 troughs.

37. The apparatus of either one of claim 35 or 36, wherein for a first lengthwise portion of the sleeve, the parallel ribs and troughs are parallel to the central longitudinal axis, and for a second lengthwise portion of the cable-sleeve the parallel ribs wind helically around the central axis.

38. The apparatus of any one of claims 33 to 37, wherein a helical pitch of the helical wire-path has a variability of less than ±50% along the length of the helical wire-path.

39. The apparatus of any one of claims 33 to 37, wherein a helical pitch of the helical wire-path is either constant or has a variability of less than ±10% along the length of the helical wire-path.

40. The apparatus of any one of claims 33 to 39, wherein a helical pitch of the helical wire-path is at least ⅓ of the length of a central-axis path of a corresponding portion of the sleeve.

41. The apparatus of any one of claims 33 to 39, wherein a helical pitch of the helical wire-path is at least ½ of the length of a central-axis path of a corresponding portion of the sleeve.

42. The apparatus of any one of claims 33 to 39, wherein a helical pitch of the helical wire-path is at most equal to 1.5 times the length of a central-axis path of a corresponding portion of the sleeve, or 1.25 times the length, or the length itself.

43. The apparatus of any one of claims 33 to 42, wherein a ratio between a helical pitch and a helical amplitude of the helical wire-path is at least 10, or at least 20, or at least 50.

44. The apparatus of any one of claims 33 to 43, wherein the bendable portion of the arm comprises a plurality of bendable segments.

45. The apparatus of any one of claims 33 to 44, wherein the bendable portion of the arm comprises non-contiguous segments.

46. The apparatus of any one of claims 33 to 45, wherein the sleeve is constrained to bend and/or straighten together with the bendable portion of the arm, and the path of the electric wire is maintained as helical relative to the bent and/or straightened path of the central longitudinal axis of the sleeve.

47. The apparatus of claim 46, wherein the path of the electric wire is maintained as helical relative to the bent and/or straightened path of the central axis of the sleeve, for any bending of the bendable portion of the arm to a radius of curvature greater than twice the diameter of the bendable portion.

48. The apparatus of claim 47, wherein the helical path of the electric wire is maintained as helical relative to the bent and/or straightened path of the central axis of the sleeve, for any bending of the bendable portion of the arm to a radius of curvature greater than 1.5 times the diameter of the bendable portion.

49. The apparatus of any one of claims 33 to 48, wherein the bending and/or straightening of the sleeve assembly does not substantially increase tension in the wire.

50. The apparatus of any one of claims 33 to 49, wherein the inner conduit has a circular cross-section.

51. The apparatus of any one of claims 33 to 50, wherein an inner diameter of the inner conduit is at least 0.5 mm and at most 2.0 mm.

52. The apparatus of any one of claims 35 to 51, wherein a maximum helical amplitude of the ribs on the outer surface of the sleeve is at least 1.0 mm and at most 2.0 mm.

53. The apparatus of any one of claims 33 to 51, wherein a maximum helical amplitude of the ribs on the outer surface of the sleeve is at least 1.25 mm and at most 1.5 mm.

54. The apparatus of any one of claims 33 to 53, wherein the surface features are formed integrally with the sleeve.

55. The apparatus of any one of claims 33 to 54, wherein the sleeve comprises a thermoplastic elastomer that includes a rigid polyamide and a flexible polyether.

56. The apparatus of any one of claims 33 to 55, wherein the electrosurgical grasper provides at least a bipolar electrosurgery mode when electrically connected to the electrosurgical power generator.

57. The apparatus of any one of claims 33 to 56, wherein the actuation-cable is mechanically coupled to the grasper such that rotation of the actuation-cable about its central axis controls the movement of at the least one jaw.

58. The apparatus of any one of claims 33 to 56, wherein the actuation-cable is mechanically coupled to the grasper such that longitudinal motion of the actuation-cable within the arm controls the movement of at the least one jaw.

59. The apparatus of any one of claims 33 to 58, additionally comprising the electrosurgical power generator, wherein an electrically conductive wire provides electrical connectivity from the power generator to the grasper.
